(19) United States
(12) Patent Application Publication
(10) Pub. No.: US 2019/0203142 A1
(43) Pub. Date: Jul. 4, 2019

(54) LUBRICATING OIL COMPOSITIONS WITH WEAR AND SLUDGE CONTROL

(71) Applicant: ExxonMobil Research and Engineering Company, Annandale, NJ (US)

(72) Inventors: Christopher VANDER NEUT, Spring, TX (US); Raymond G. BURNS, III, Easton, PA (US)

(21) Appl. No.: 16/212,164

(22) Filed: Dec. 6, 2018

Related U.S. Application Data
(60) Provisional application No. 62/611,576, filed on Dec. 29, 2017.

(51) Int. Cl. C10M 129/40; C10M 169/04; G01N 33/28

(52) U.S. Cl. CPC C10M 129/40; C10M 169/04; G01N 33/2882; C10M 2203/003; C10M 2207/126; C10N 2240/04; C10N 2230/04; C10N 2230/54; C10N 2210/04; C10N 2240/10; C10N 2240/08; C10N 2230/06

(57) ABSTRACT

A method for improving wear control and sludge control, while maintaining or improving fuel efficiency, of a lubricating oil in an engine or other mechanical component lubricated with the lubricating oil by using as the lubricating oil a formulated oil. The formulated oil has a composition including a lubricating oil base stock as a major component, and at least one lubricating oil additive, as a minor component. The at least one lubricating oil additive includes a zirconium-containing compound. The zirconium-containing compound is present in an amount from about 0.1 to about 1200 parts per million (ppm). The zirconium-containing compound is soluble in the lubricating oil base stock. The lubricating oil is useful as a passenger vehicle engine oil (PVEO), a commercial vehicle engine oil (CVEO), and other lubricating oils (hydraulic, gear, transmission, etc.).

### CROSS-REFERENCE TO RELATED APPLICATIONS

This application claims the benefit of U.S. Provisional Application No. 62/611,576, filed on December 29, 2017, the entire contents of which are incorporated herein by reference.

### FIELD

This disclosure relates to engine lubricating oils with wear control and sludge control. In particular, this disclosure relates to lubricating oils, methods for improving wear control and sludge control, while maintaining or improving fuel efficiency, of a lubricating oil in an engine or other mechanical component lubricated with the lubricating oil, and methods for authenticating lubricating oils (e.g., for product quality control, anti-counterfeit protection, and genuine product verification). The lubricating oils of this disclosure are useful as passenger vehicle engine oil (PVEO) products, or commercial vehicle engine oil (CVEO) products and other lubricating oils (hydraulic, gear, transmission, etc.).

### BACKGROUND

Lubricant-related performance characteristics such as high temperature wear protection and sludge control, and fuel economy are extremely advantageous attributes as measured by a variety of bench and engine tests.

Lubricant-related wear control is highly desirable due to increasing use of low viscosity working fluids for improved fuel efficiency. As governmental regulations for vehicle fuel consumption and carbon emissions become more stringent, use of low viscosity lubricantsoils to meet the regulatory standards is becoming more prevalent. At the same time, lubricants need to provide a substantial level of durability and wear protection due to the formation of thinner lubricant films during engine operation. As such, use of antiwear additives and friction modifiers in a lubricant formulation is the typical method for achieving wear control and durability. Due to limitations of using high levels of antiwear and friction modifier additives such as catalyst poisoning and sludge formation, it is highly desirable to find alternative methods for achieving excellent wear control and durability.

Current state of the art for antiwear improvements involve the use of either traditional zinc dialkyl dithio phosphate (ZDDP) or of ashless antiwear additives. The current art for sludge control teaches that detergents are the ideal solution to improve sludge performance.

A major challenge in lubricant formulation is simultaneously achieving high temperature wear control and sludge control, while also maintaining or improving fuel economy.

Despite the advances in lubricant oil formulation technology, there exists a need for newly designed lubricants that effectively improve wear control and sludge control while maintaining or improving fuel efficiency.

### SUMMARY

This disclosure relates to working fluids with wear control and sludge control. In particular, this disclosure relates to lubricating oils, methods for improving wear control and sludge control, while maintaining or improving fuel efficiency, of a lubricating oil in an engine or other mechanical component lubricated with the lubricating oil, and methods for authenticating lubricating oils (e.g., for product quality control, anti-counterfeit protection, and genuine product verification). The lubricating oils of this disclosure are useful as passenger vehicle engine oil (PVEO) products, commercial vehicle engine oil (CVEO) products and other lubricating oils (hydraulic, gear, transmission, etc.).

This disclosure also relates in part to a method for improving wear control and sludge control, while maintaining or improving fuel efficiency, of a lubricating oil in an engine or other mechanical component lubricated with the lubricating oil by using as the lubricating oil a formulated oil. The formulated oil has a composition comprising a lubricating oil base stock as a major component, and at least one lubricating oil additive, as a minor component. The at least one lubricating oil additive comprises a zirconium-containing compound. The zirconium-containing compound is present in an amount from about 0.1 to about 1200 parts per million (ppm). The zirconium-containing compound is soluble in the lubricating oil base stock.

This disclosure further relates in part to a lubricating oil having a lubricating oil base stock as a major component, and at least one lubricating oil additive, as a minor component. The at least one lubricating oil additive comprises a zirconium-containing compound. The zirconium-containing compound is present in an amount from about 0.1 to about 1200 parts per million (ppm). The zirconium-containing compound is soluble in the lubricating oil base stock.

This disclosure yet further relates in part to a method for authenticating a lubricating oil. The method comprises (i) marking the lubricating oil by introducing at least one metallic tracer into the lubricating oil, (ii) optionally lubricating an engine or other mechanical component with the lubricating oil, and (iii) authenticating the lubricating oil by determining at least one of the identity and amount of the at least one metallic tracer in the lubricating oil. The lubricating oil comprises a lubricating oil base stock, and the at least one metallic tracer comprises a zirconium-containing compound. The zirconium-containing compound is present in an amount from about 0.1 to about 1200 parts per million (ppm). The zirconium-containing compound is soluble in the lubricating oil base stock.

It has been surprisingly found that, in wear performance measurements of the lubricating oil using a Four Ball Wear Test in accordance with ASTM D4172, the wear scar diameter in millimeters (mm) for the lubricating oil having a zirconium treat rate from about 0.1 to about 1200 parts per million (ppm) is decreased as compared to the wear scar diameter (mm) of a lubricating oil having a zirconium treat rate of 0 ppm.

Also, it has been surprisingly found that, in sludge control measurements of the lubricating oil using the B-10 Catalytic Oxidation Test, the sludge rating for the lubricating oil having a zirconium treat rate from about 0.1 to about 1200 parts per million (ppm) is decreased as compared to the sludge rating of a lubricating oil having a zirconium treat rate of 0 ppm, in which the sludge rating is based on a scale of 0 (nil), 1 (trace), 2 (light), and 3 (heavy).

Other objects and advantages of the present disclosure will become apparent from the detailed description that follows.

### BRIEF DESCRIPTION OF THE DRAWINGS

FIG. 1 shows formulation details for relevant blends in EHC 65, and shows reference oil as neat EHC 65 and comparative blends as varying concentrations of zirconium 2-ethylhexanoate blended with neat EHC 65 base stock, in accordance with the Examples.

FIG. 2 shows formulation details for relevant blends in formulated 15W-40 heavy duty engine oil, and shows reference oil as fully formulated commercial 15W-40 heavy duty engine oil and comparative blends as varying concentrations of zirconium 2-ethylhexanoate blended into fully formulated oil in balance with EHC 65 base stock, in accordance with the Examples.

FIG. 3 shows physical properties for relevant blends, and shows viscosity and relevant additive metals in tested formulations, in accordance with the Examples.

FIG. 4 graphically shows ASTM D4172 Four Ball Wear Test results at medium and high load for reference and comparative blends in neat EHC 65 base stock, in accordance with the Examples.

FIG. 5 graphically shows B-10 Sludge Performance by AM/S 334 in Blend Series B, in which Blend Series B is a fully formulated heavy duty diesel engine oil, in accordance with the Examples.

### DETAILED DESCRIPTION

All numerical values within the detailed description and the claims herein are modified by “about” or “approximately” the indicated value, and take into account experimental error and variations that would be expected by a person having ordinary skill in the art. The phrase “major amount” or “major component” as it relates to components included within the lubricating oils of the specification and the claims means greater than or equal to 50 wt. %, or greater than or equal to 60 wt. %, or greater than or equal to 70 wt. %, or greater than or equal to 80 wt. %, or greater than or equal to 90 wt. % based on the total weight of the lubricating oil. The phrase “minor amount” or “minor component” as it relates to components included within the lubricating oils of the specification and the claims means less than 50 wt. %, or less than or equal to 40 wt. %, or less than or equal to 30 wt. %, or greater than or equal to 20 wt. %, or less than or equal to 10 wt. %, or less than or equal to 5 wt. %, or less than or equal to 2 wt. %, or less than or equal to 1 wt. %, based on the total weight of the lubricating oil. The phrase “essentially free” as it relates to components included within the lubricating oils of the specification and the claims means that the particular component is at 0 weight % within the lubricating oil, or alternatively is at impurity type levels within the lubricating oil (less than 100 ppm, or less than 20 ppm, or less than 10 ppm, or less than 1 ppm). The phrase “other lubricating oil additives” as used in the specification and the claims means other lubricating oil additives that are not specifically recited in the particular section of the specification or the claims. For example, other lubricating oil additives may include, but are not limited to, antioxidants, detergents, dispersants, antiwear additives, corrosion inhibitors, viscosity modifiers, metal passivators, pour point depressants, seal compatibility agents, antifoam agents, extreme pressure agents, friction modifiers and combinations thereof

In accordance with this disclosure, zirconium-containing compounds (e.g., zirconium 2-ethylhexanoate) are used as antiwear components in lubricating oils. Benefits have been surprisingly discovered in the use of zirconium-containing compounds for wear performance and sludge protection. Even small amounts of zirconium-containing compounds (e.g., zirconium 2-ethylhexanoate) produce improvements in the Four Ball Wear Test (ASTM D4172) under normal and high load conditions compared to EHC 65.

Also, small amounts of zirconium-containing compounds have been added to fully formulated heavy duty diesel engine oils and evaluated in AM/S 334, commonly known as the B-10 Catalytic Oxidation Test, described herein. The results of this test under two temperatures showed an improvement in sludge performance when using zirconium 2-ethylhexanoate.

The zirconium-containing compounds (e.g., zirconium 2-ethylhexanoate) can also be used as a tracer. In particular, the zirconium-containing compounds (e.g., zirconium 2-ethylhexanoate) can be added to commercial products in support of quality control, anti- counterfitting, and genuine product verification through fresh oil and used oil metals testing.

This disclosure provides a simple means to improve wear and sludge performance, and also mark the lubricating oil in a known way for traceability. This is accomplished in low concentrations, between 1-500 ppm of zirconium (Zr), and does not cause any detrimental performance in other performance parameters such as oxidation, filterability, demulsibility, or low temperature performance. Benefits are observed in both neat base oils, as well as fully formulated lubricating oils. Other viable tracers or unique antiwear components can cause negative effects in other performance areas.

The zirconium tracer materials of this disclosure can survive in harsh lubricating oil environments, either in industrial applications, fired engines, or other extreme temperature, pressure, shear, acidity, environments, and the like. The use of zirconium-containing compounds (e.g., zirconium 2-ethylhexanoate) enables entry into this tracer space, while also providing a benefit in the antiwear and sludge performance of the oil. An important benefit provided by the zirconium-containing compounds (e.g., zirconium 2-ethylhexanoate) is their enhancement of the oil antiwear and sludge capability.

Current state of the art for antiwear improvements involve the use of either traditional ZDDP or of ashless antiwear additives. This disclosure provides a new antiwear solution, namely zirconium-containing compounds (e.g., zirconium 2-ethylhexanoate) for use at low concentrations. The current art for sludge control teaches that detergents are the ideal solution to improve sludge performance. This disclosure shows that zirconium-containing compounds (e.g., zirconium 2-ethylhexanoate) can be used in fully formulated working fluids to improve sludge performance.

Also, current state of the art for lubricant tracers includes either fluorescent dyes or other more subtle markers that require sophisticated lab equipment like GC-MS. This disclosure simply provides a metallic tracer, which employs zirconium, a metal not commonly found in lubricant additives or machine hardware and metallurgy. This makes it an ideal candidate for used oil identification.

This disclosure enables the use of low concentrations of zirconium-containing compound (e.g., zirconium 2-ethylhexanoate) additives in order to improve antiwear and sludge performance, and also enables the use a zirconium tracer for product identification and authentication. This simple solution is able to be employed at low concentrations, making it a low cost option for an improved formulation.

### Lubricating Oil Base Stocks

A wide range of lubricating base oils is known in the art. Lubricating base oils that are useful in the present disclosure are both natural oils, and synthetic oils, and unconventional oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve at least one lubricating oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad base oil stock categories developed and defined by the American Petroleum Institute (API Publication 1509; www.API.org) to create guidelines for lubricant base oils. Group I base stocks have a viscosity index of between about 80 to 120 and contain greater than about 0.03% sulfur and/or less than about 90% saturates. Group II base stocks have a viscosity index of between about 80 to 120, and contain less than or equal to about 0.03% sulfur and greater than or equal to about 90% saturates. Group III stocks have a viscosity index greater than about 120 and contain less than or equal to about 0.03% sulfur and greater than about 90% saturates. Group IV includes polyalphaolefins (PAO). Group V base stock includes base stocks not included in Groups I-IV. The table below summarizes properties of each of these five groups.

| | Base Oil Properties | | |
|---|---|---|---|
| | Saturates | Sulfur | Viscosity Index |
| Group I | <90 and/or | >0.03% and | ≥80 and <120 |
| Group II | ≥90 and | ≤0.03% and | ≥80 and <120 |
| Group III | ≥90 and | ≤0.03% and | ≥120 |

| | |
|---|---|
| Group IV | Polyalphaolefins (PAO) |
| Group V | All other base oil stocks not included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful. Natural oils vary also as to the method used for their production and purification, for example, their distillation range and whether they are straight run or cracked, hydrorefined, or solvent extracted.

Group II and/or Group III hydroprocessed or hydrocracked base stocks, including synthetic oils such as polyalphaolefins, alkyl aromatics and synthetic esters are also well known base stock oils.

Synthetic oils include hydrocarbon oil. Hydrocarbon oils include oils such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks are commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073.

The number average molecular weights of the PAOs, which are known materials and generally available on a major commercial scale from suppliers such as ExxonMobil Chemical Company, Chevron Phillips Chemical Company, BP, and others, typically vary from about 250 to about 3,000, although PAO's may be made in viscosities up to about 150 cSt (100° C.). The PAOs are typically comprised of relatively low molecular weight hydrogenated polymers or oligomers of alphaolefins which include, but are not limited to, C₂ to about C₃₂ alphaolefins with the C₈ to about C₁₆ alphaolefins, such as 1-hexene, 1-octene, 1-decene, 1-dodecene and the like, being preferred. The preferred polyalphaolefins are poly1-hexene, poly-1-octene, poly-1-decene and poly-l-dodecene and mixtures thereof and mixed olefin-derived polyolefins. However, the dimers of higher olefins in the range of C₁₄ to C₁₈ may be used to provide low viscosity base stocks of acceptably low volatility. Depending on the viscosity grade and the starting oligomer, the PAOs may be predominantly trimers and tetramers of the starting olefins, with minor amounts of the higher oligomers, having a viscosity range of 1.5 to 12 cSt. PAO fluids of particular use may include 3.0 cSt, 3.4 cSt, and/or 3.6 cSt and combinations thereof. Bi-modal mixtures of PAO fluids having a viscosity range of 1.5 to 150 cSt may be used if desired.

The PAO fluids may be conveniently made by the polymerization of an alphaolefin in the presence of a polymerization catalyst such as the Friedel-Crafts catalysts including, for example, aluminum trichloride, boron trifluoride or complexes of boron trifluoride with water, alcohols such as ethanol, propanol or butanol, carboxylic acids or esters such as ethyl acetate or ethyl propionate. For example the methods disclosed by U.S. Pat. Nos. 4,149,178 or 3,382,291 may be conveniently used herein. Other descriptions of PAO synthesis are found in the following U.S. Pat. Nos. 3,742,082; 3,769,363; 3,876,720; 4,239,930; 4,367,352; 4,413,156; 4,434,408; 4,910,355; 4,956,122; and 5,068,487. The dimers of the C₁₄ to C₁₈ olefins are described in U.S. Pat. No. 4,218,330.

The alkylated naphthalene can be used as base oil or base oil component and can be any hydrocarbyl molecule that contains at least about 5% of its weight derived from a naphthenoid moiety, or its derivatives. These alkylated naphthalenes include alkyl naphthalenes, alkyl naphthols, and the like. The naphthenoid group can be mono-alkylated, dialkylated, polyalkylated, and the like. The naphthenoid group can be mono- or poly-functionalized. The naphthenoid group can also be derived from natural (petroleum) sources, provided at least about 5% of the molecule is comprised of the naphthenoid moiety. Viscosities at 100° C. of approximately 3 cSt to about 50 cSt are preferred, with viscosities of approximately 3.4 cSt to about 20 cSt often being more preferred for the naphthylene component. In one embodiment, an alkyl naphthalene where the alkyl group is primarily comprised of 1-hexadecene is used. Other alkylates of naphthalene can be advantageously used. Naphthalene or methyl naphthalene, for example, can be alkylated with olefins such as octene, decene, dodecene, tetradecene or higher, mixtures of similar olefins, and the like.

Alkylated naphthalenes of the present disclosure may be produced by well-known Friedel-Crafts alkylation of aromatic compounds. See Friedel-Crafts and Related Reactions, Olah, G. A. (ed.), Inter-science Publishers, N.Y., 1963. For example, an aromatic compound, such as naphthalene, is alkylated by an olefin, alkyl halide or alcohol in the presence of a Friedel-Crafts catalyst. See Friedel-Crafts and Related Reactions, Vol. 2, part 1, chapters 14, 17, and 18, See Olah, G. A. (ed.), Inter-science Publishers, N.Y., 1964. Many homogeneous or heterogeneous, solid catalysts are known to one skilled in the art. The choice of catalyst depends on the reactivity of the starting materials and product quality requirements. For example, strong acids such as AlCl₃, BF₃, or HF may be used. In some cases, milder catalysts such as FeCl3 or SnCl₄ are preferred. Newer alkylation technology uses zeolites or solid super acids.

Mixtures of alkylated naphthalene base stocks with other lubricating oil base stocks (e.g., Groups I, II, III, IV and V base stocks) may be useful in the lubricating oil formulations of this disclosure.

The alkylated naphthalene can be present in an amount of from about 30 to about 99.8 weight percent, or from about 35 to about 95 weight percent, or from about 40 to about 90 weight percent, or from about 45 to about 85 weight percent, or from about 50 to about 80 weight percent, or from about 55 to about 75 weight percent, or from about 60 to about 70 weight percent, based on the total weight of the formulated oil.

Other useful lubricant oil base stocks include wax isomerate base stocks and base oils, comprising hydroisomerized waxy stocks (e.g. waxy stocks such as gas oils, slack waxes, fuels hydrocracker bottoms, etc.), hydroisomerized Fischer-Tropsch waxes, Gas-to-Liquids (GTL) base stocks and base oils, and other wax isomerate hydroisomerized base stocks and base oils, or mixtures thereof Fischer-Tropsch waxes, the high boiling point residues of Fischer-Tropsch synthesis, are highly paraffinic hydrocarbons with very low sulfur content. The hydroprocessing used for the production of such base stocks may use an amorphous hydrocracking/hydroisomerization catalyst, such as one of the specialized lube hydrocracking (LHDC) catalysts or a crystalline hydrocracking/hydroisomerization catalyst, preferably a zeolitic catalyst. For example, one useful catalyst is ZSM-48 as described in U.S. Pat. No. 5,075,269, the disclosure of which is incorporated herein by reference in its entirety. Processes for making hydrocracked/hydroisomerized distillates and hydrocracked/hydroisomerized waxes are described, for example, in U.S. Pat. Nos. 2,817,693; 4,975,177; 4,921,594 and 4,897,178 as well as in British Patent Nos. 1,429,494; 1,350,257; 1,440,230 and 1,390,359. Each of the aforementioned patents is incorporated herein in their entirety. Particularly favorable processes are described in European Patent Application Nos. 464546 and 464547, also incorporated herein by reference. Processes using Fischer-Tropsch wax feeds are described in U.S. Pat. Nos. 4,594,172 and 4,943,672, the disclosures of which are incorporated herein by reference in their entirety.

Gas-to-Liquids (GTL) base oils, Fischer-Tropsch wax derived base oils, and other wax-derived hydroisomerized (wax isomerate) base oils be advantageously used in the instant disclosure, and may have useful kinematic viscosities at 100° C. of about 3 cSt to about 50 cSt, preferably about 3 cSt to about 30 cSt, more preferably about 3.5 cSt to about 25 cSt, as exemplified by GTL 4 with kinematic viscosity of about 4.0 cSt at 100° C. and a viscosity index of about 141. These Gas-to-Liquids (GTL) base oils, Fischer-Tropsch wax derived base oils, and other wax-derived hydroisomerized base oils may have useful pour points of about -20° C. or lower, and under some conditions may have advantageous pour points of about −25° C. or lower, with useful pour points of about -30° C. to about -40° C. or lower. Useful compositions of Gas-to-Liquids (GTL) base oils, Fischer-Tropsch wax derived base oils, and wax-derived hydroisomerized base oils are recited in U.S. Patent Nos. 6,080,301; 6,090,989, and 6,165,949 for example, and are incorporated herein in their entirety by reference.

The hydrocarbyl aromatics can be used as base oil or base oil component and can be any hydrocarbyl molecule that contains at least about 5% of its weight derived from an aromatic moiety such as a benzenoid moiety or naphthenoid moiety, or their derivatives. These hydrocarbyl aromatics include alkyl benzenes, alkyl naphthalenes, alkyl diphenyl oxides, alkyl naphthols, alkyl diphenyl sulfides, alkylated bis-phenol A, alkylated thiodiphenol, and the like. The aromatic can be mono-alkylated, dialkylated, polyalkylated, and the like. The aromatic can be mono- or poly-functionalized. The hydrocarbyl groups can also be comprised of mixtures of alkyl groups, alkenyl groups, alkynyl, cycloalkyl groups, cycloalkenyl groups and other related hydrocarbyl groups. The hydrocarbyl groups can range from about C₆ up to about C₆₀ with a range of about C₈ to about C₂₀ often being preferred. A mixture of hydrocarbyl groups is often preferred, and up to about three such substituents may be present. The hydrocarbyl group can optionally contain sulfur, oxygen, and/or nitrogen containing substituents. The aromatic group can also be derived from natural (petroleum) sources, provided at least about 5% of the molecule is comprised of an above-type aromatic moiety. Viscosities at 100° C. of approximately 3 cSt to about 50 cSt are preferred, with viscosities of approximately 3.4 cSt to about 20 cSt often being more preferred for the hydrocarbyl aromatic component. In one embodiment, an alkyl naphthalene where the alkyl group is primarily comprised of 1-hexadecene is used. Other alkylates of aromatics can be advantageously used. Naphthalene or methyl naphthalene, for example, can be alkylated with olefins such as octene, decene, dodecene, tetradecene or higher, mixtures of similar olefins, and the like. Useful concentrations of hydrocarbyl aromatic in a lubricant oil composition can be about 2% to about 25%, preferably about 4% to about 20%, and more preferably about 4% to about 15%, depending on the application.

Alkylated aromatics such as the hydrocarbyl aromatics of the present disclosure may be produced by well-known Friedel-Crafts alkylation of aromatic compounds. See Friedel-Crafts and Related Reactions, Olah, G. A. (ed.), Inter-science Publishers, N.Y., 1963. For example, an aromatic compound, such as benzene or naphthalene, is alkylated by an olefin, alkyl halide or alcohol in the presence of a Friedel-Crafts catalyst. See Friedel-Crafts and Related Reactions, Vol. 2, part 1, chapters 14, 17, and 18, See Olah, G. A. (ed.), Inter-science Publishers, N.Y., 1964. Many homogeneous or heterogeneous, solid catalysts are known to one skilled in the art. The choice of catalyst depends on the reactivity of the starting materials and product quality requirements. For example, strong acids such as AlC₁₃, BF₃, or H may be used. In some cases, milder catalysts such as FeCl₃ or SnCl₄ are preferred. Newer alkylation technology uses zeolites or solid super acids.

Esters comprise a useful base stock. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, alkyl succinic acid, alkenyl succinic acid, maleic acid, azelaic acid, suberic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols (such as the neopentyl polyols, e.g., neopentyl glycol, trimethylol ethane, 2-methyl-2-propyl-1,3-propanediol, trimethylol propane, pentaerythritol and dipentaerythritol) with alkanoic acids containing at least about 4 carbon atoms, preferably C₅ to C₃₀ acids such as saturated straight chain fatty acids including caprylic acid, capric acid, lauric acid, myristic acid, palmitic acid, stearic acid, arachic acid, and behenic acid, or the corresponding branched chain fatty acids or unsaturated fatty acids such as oleic acid, or mixtures of any of these materials.

Suitable synthetic ester components include the esters of trimethylol propane, trimethylol butane, trimethylol ethane, pentaerythritol and/or dipentaerythritol with one or more monocarboxylic acids containing from about 5 to about 10 carbon atoms. These esters are widely available commercially, for example, the Mobil P-41 and P-51 esters of ExxonMobil Chemical Company.

Also useful are esters derived from renewable material such as coconut, palm, rapeseed, soy, sunflower and the like. These esters may be monoesters, di-esters, polyol esters, complex esters, or mixtures thereof. These esters are widely available commercially, for example, the Mobil P-51 ester of ExxonMobil Chemical Company.

Engine oil formulations containing renewable esters are included in this disclosure. For such formulations, the renewable content of the ester is typically greater than about 70 weight percent, preferably more than about 80 weight percent and most preferably more than about 90 weight percent.

Other useful fluids of lubricating viscosity include non-conventional or unconventional base stocks that have been processed, preferably catalytically, or synthesized to provide high performance lubrication characteristics.

Non-conventional or unconventional base stocks/base oils include one or more of a mixture of base stock(s) derived from one or more Gas-to-Liquids (GTL) materials, as well as isomerate/isodewaxate base stock(s) derived from natural wax or waxy feeds, mineral and or non-mineral oil waxy feed stocks such as slack waxes, natural waxes, and waxy stocks such as gas oils, waxy fuels hydrocracker bottoms, waxy raffinate, hydrocrackate, thermal crackates, or other mineral, mineral oil, or even non-petroleum oil derived waxy materials such as waxy materials received from coal liquefaction or shale oil, and mixtures of such base stocks.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; (3) hydrodewaxed or hydroisomerized cat and/or solvent dewaxed Fischer-Tropsch (F-T) material (i.e., hydrocarbons, waxy hydrocarbons, waxes and possible analogous oxygenates); preferably hydrodewaxed or hydroisomerized/followed by cat and/or solvent dewaxing dewaxed F-T waxy hydrocarbons, or hydrodewaxed or hydroisomerized/followed by cat (or solvent) dewaxing dewaxed, F-T waxes, or mixtures thereof

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having kinematic viscosities at 100° C. of from about 2 mm²/s to about 50 mm²/s (ASTM D445). They are further characterized typically as having pour points of −5° C. to about −40° C. or lower (ASTM D97). They are also characterized typically as having viscosity indices of about 80 to about 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than about 10 ppm, and more typically less than about 5 ppm of each of these elements. The sulfur and nitrogen content of GTL base stock(s) and/or base oil(s) obtained from F-T material, especially F-T wax, is essentially nil. In addition, the absence of phosphorous and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the formulated lubricating oils useful in the present disclosure are any of the variety of oils corresponding to API Group I, Group II, Group III, Group IV, and Group V oils and mixtures thereof, preferably API Group II, Group III, Group IV, and Group V oils and mixtures thereof, more preferably the Group III to Group V base oils due to their exceptional volatility, stability, viscometric and cleanliness features.

The base oil constitutes the major component of the lubricant composition of the present disclosure and typically is present in an amount ranging from about 50 to about 99 weight percent, preferably from about 70 to about 95 weight percent, and more preferably from about 85 to about 95 weight percent, based on the total weight of the composition. The base oil may be selected from any of the synthetic or natural oils typically used as crankcase lubricating oils for spark-ignited and compression-ignited engines. The base oil conveniently has a kinematic viscosity, according to ASTM standards, of about 2.5 cSt to about 12 cSt (or mm² /s) at 100° C. and preferably of about 2.5 cSt to about 9 cSt (or mm² /s) at 100° C. Mixtures of synthetic and natural base oils may be used if desired. Bi-modal mixtures of Group I, II, III, IV, and/or V base stocks may be used if desired.

### Zirconium-Containing Compounds

A wide range of zirconium-containing compounds can be used in the lubricating oils of this disclosure. The zirconium-containing compounds are soluble in the lubricating oil base stocks, and are used in low concentrations.

Illustrative zirconium-containing compounds include, for example, zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, zirconium salts, and the like. Illustrative zirconium salts include, for example, zirconium oleate, zirconium stearate, zirconium palmitate, zirconium laurate, and the like.

The preferred zirconium-containing compound is zirconium 2-ethylhexanoate.

In accordance with this disclosure, an important benefit provided by the zirconium-containing compounds (e.g., zirconium 2-ethylhexanoate) is their enhancement of the oil antiwear and sludge capability. Another important benefit provided by the zirconium-containing compounds (e.g., zirconium 2-ethylhexanoate) is tracer applications and their ability to survive in harsh lubricating oil environments, either in industrial applications, fired engines, or other extreme temperature, pressure, shear, acidity, environments, and the like. The use of zirconium-containing compounds (e.g., zirconium 2-ethylhexanoate) provides benefits in the antiwear and sludge performance of the lubricating oil, and also benefits in tracer applications.

This disclosure enables the use of low concentrations of zirconium-containing compound (e.g., zirconium 2-ethylhexanoate) additives in order to improve antiwear and sludge performance, and also enables the use of low concentrations of zirconium tracer for product identification and authentication. These solutions are able to be achieved at low concentrations, making the low concentrations a low cost option for improved formulations.

The zirconium-containing compounds are present in the lubricating oils of this disclosure in an amount from about 0.1 to about 1200 parts per million (ppm), preferably from about 1 to about 1000 parts per million (ppm), and more preferably from about 10 to about 800 parts per million (ppm). For tracer applications, the zirconium-containing compounds preferably are present in an amount from about 1 to about 500 parts per million (ppm) more preferably from about 10 to about 250 ppm, still more preferably from about 50 to about 200 ppm, further more preferably from about 50 to about 100 ppm.

### Lubricating Oil Additives

The formulated lubricating oil useful in the present disclosure may additionally contain one or more of the commonly used lubricating oil performance additives including but not limited to antioxidants, dispersants, detergents, antiwear additives, corrosion inhibitors, rust inhibitors, metal deactivators, extreme pressure additives, anti-seizure agents, wax modifiers, viscosity index improvers, viscosity modifiers, fluid-loss additives, seal compatibility agents, friction modifiers, lubricity agents, anti-staining agents, chromophoric agents, defoamants, demulsifiers, emulsifiers, densifiers, wetting agents, gelling agents, tackiness agents, colorants, and others. For a review of many commonly used additives, see Klamann in Lubricants and Related Products, Verlag Chemie, Deerfield Beach, Fla.; ISBN 0-89573-177-0. Reference is also made to “Lubricant Additives” by

M. W. Ranney, published by Noyes Data Corporation of Parkridge, N.J. (1973); see also U.S. Pat. No. 7,704,930, the disclosure of which is incorporated herein in its entirety. These additives are commonly delivered with varying amounts of diluent oil, that may range from 5 weight percent to 50 weight percent.

### Antioxidants

Antioxidants retard the oxidative degradation of base oils during service. Such degradation may result in deposits on metal surfaces, the presence of sludge, or a viscosity increase in the lubricant. One skilled in the art knows a wide variety of oxidation inhibitors that are useful in lubricating oil compositions. See, Klamann in Lubricants and Related Products, op cite, and U.S. Pat. Nos. 4,798,684 and 5,084,197, for example.

Useful antioxidants include hindered phenols. These phenolic antioxidants may be ashless (metal-free) phenolic compounds or neutral or basic metal salts of certain phenolic compounds. Typical phenolic antioxidant compounds are the hindered phenolics which are the ones which contain a sterically hindered hydroxyl group, and these include those derivatives of dihydroxy aryl compounds in which the hydroxyl groups are in the o- or p-position to each other.

Typical phenolic antioxidants include the hindered phenols substituted with C₆₊ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; and 2-methyl-6-t-butyl-4-dodecyl phenol. Other useful hindered mono-phenolic antioxidants may include for example hindered 2,6-di-alkyl-phenolic proprionic ester derivatives. Bis-phenolic antioxidants may also be advantageously used in combination with the instant disclosure. Examples of ortho-coupled phenols include: 2,2′-bis(4-heptyl-6-t-butyl-phenol); 2,2′-bis(4-octyl- and 2,2′-bis(4-dodecyl-6-t-butyl-phenol). Para-coupled bisphenols include for example 4,4′-bis(2,6-di-t-butyl phenol) and 4,4′-methylene-bis(2,6-di-t-butyl phenol).

Effective amounts of one or more catalytic antioxidants may also be used. The catalytic antioxidants comprise an effective amount of a) one or more oil soluble polymetal organic compounds; and, effective amounts of b) one or more substituted N,N′-diaryl-o-phenylenediamine compounds or c) one or more hindered phenol compounds; or a combination of both b) and c). Catalytic antioxidants are more fully described in U.S. Pat. No. 8, 048,833, herein incorporated by reference in its entirety.

Non-phenolic oxidation inhibitors which may be used include aromatic amine antioxidants and these may be used either as such or in combination with phenolics. Typical examples of non-phenolic antioxidants include: alkylated and non-alkylated aromatic amines such as aromatic monoamines of the formula R⁸R⁹R¹N where R⁸ is an aliphatic, aromatic or substituted aromatic group, R⁹ is an aromatic or a substituted aromatic group, and R¹⁰ is H, alkyl, aryl or R¹¹S(O)xR¹² where R¹¹ is an alkylene, alkenylene, or aralkylene group,R¹² is a higher alkyl group, or an alkenyl, aryl, or alkaryl group, and x is 0, 1 or 2. The aliphatic group R⁸ may contain from 1 to about 20 carbon atoms, and preferably contains from about 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines antioxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of amine antioxidants useful in the present compositions include diphenylamines, phenyl naphthylamines, phenothiazines, imidodibenzyls and diphenyl phenylene diamines. Mixtures of two or more aromatic amines are also useful. Polymeric amine antioxidants can also be used. Particular examples of aromatic amine antioxidants useful in the present disclosure include: p,p′-dioctyldiphenylamine; t-octylphenyl-alpha-naphthylamine; phenyl-alphanaphthylamine; and p-octylphenyl-alpha-naphthylamine.

Sulfurized alkyl phenols and alkali or alkaline earth metal salts thereof also are useful antioxidants.

Preferred antioxidants include hindered phenols, arylamines. These antioxidants may be used individually by type or in combination with one another. Such additives may be used in an amount of about 0.01 to 5 weight percent, preferably about 0.01 to 1.5 weight percent, more preferably zero to less than 1.5 weight percent, more preferably zero to less than 1 weight percent.

### Dispersants

During engine operation, oil-insoluble oxidation byproducts are produced. Dispersants help keep these byproducts in solution, thus diminishing their deposition on metal surfaces. Dispersants used in the formulation of the lubricating oil may be ashless or ash-forming in nature. Preferably, the dispersant is ashless. So called ashless dispersants are organic materials that form substantially no ash upon combustion. For example, non-metal-containing or borated metal-free dispersants are considered ashless. In contrast, metal-containing detergents discussed above form ash upon combustion.

Suitable dispersants typically contain a polar group attached to a relatively high molecular weight hydrocarbon chain. The polar group typically contains at least one element of nitrogen, oxygen, or phosphorus. Typical hydrocarbon chains contain 50 to 400 carbon atoms.

A particularly useful class of dispersants are the (poly)alkenylsuccinic derivatives, typically produced by the reaction of a long chain hydrocarbyl substituted succinic compound, usually a hydrocarbyl substituted succinic anhydride, with a polyhydroxy or polyamino compound. The long chain hydrocarbyl group constituting the oleophilic portion of the molecule which confers solubility in the oil, is normally a polyisobutylene group. Many examples of this type of dispersant are well known commercially and in the literature. Exemplary U.S. patents describing such dispersants are U.S. Pat. Nos. 3,172,892; 3,2145,707; 3,219,666; 3,316,177; 3,341,542; 3,444,170; 3,454,607; 3,541,012; 3,630,904; 3,632,511; 3,787,374 and 4,234,435. Other types of dispersant are described in U.S. Pat. Nos. 3,036,003; 3,200,107; 3,254,025; 3,275,554; 3,438,757; 3,454,555; 3,565,804; 3,413,347; 3,697,574; 3,725,277; 3,725,480; 3,726,882; 4,454,059; 3,329,658; 3,449,250; 3,519,565; 3,666,730; 3,687,849; 3,702,300; 4,100,082; 5,705,458. A further description of dispersants may be found, for example, in European Patent Application No. 471 071, to which reference is made for this purpose.

Hydrocarbyl-substituted succinic acid and hydrocarbyl-substituted succinic anhydride derivatives are useful dispersants. In particular, succinimide, succinate esters, or succinate ester amides prepared by the reaction of a hydrocarbon-substituted succinic acid compound preferably having at least 50 carbon atoms in the hydrocarbon substituent, with at least one equivalent of an alkylene amine are particularly useful.

Succinimides are formed by the condensation reaction between hydrocarbyl substituted succinic anhydrides and amines. Molar ratios can vary depending on the polyamine. For example, the molar ratio of hydrocarbyl substituted succinic anhydride to TEPA can vary from about 1:1 to about 5:1. Representative examples are shown in U.S. Pat. Nos. 3,087,936; 3,172,892; 3,219,666; 3,272,746; 3,322,670; and 3,652,616, 3,948,800; and Canada Patent No. 1,094,044.

Succinate esters are formed by the condensation reaction between hydrocarbyl substituted succinic anhydrides and alcohols or polyols. Molar ratios can vary depending on the alcohol or polyol used. For example, the condensation product of a hydrocarbyl substituted succinic anhydride and pentaerythritol is a useful dispersant.

Succinate ester amides are formed by condensation reaction between hydrocarbyl substituted succinic anhydrides and alkanol amines. For example, suitable alkanol amines include ethoxylated polyalkylpolyamines, propoxylated polyalkylpolyamines and polyalkenylpolyamines such as polyethylene polyamines. One example is propoxylated hexamethylenediamine. Representative examples are shown in U.S. Pa. No. 4,426,305.

The molecular weight of the hydrocarbyl substituted succinic anhydrides used in the preceding paragraphs will typically range between 800 and 2,500 or more. The above products can be post-reacted with various reagents such as sulfur, oxygen, formaldehyde, carboxylic acids such as oleic acid. The above products can also be post reacted with boron compounds such as boric acid, borate esters or highly borated dispersants, to form borated dispersants generally having from about 0.1 to about 5 moles of boron per mole of dispersant reaction product.

Mannich base dispersants are made from the reaction of alkylphenols, formaldehyde, and amines. See U.S. Pat. No. 4,767,551, which is incorporated herein by reference. Process aids and catalysts, such as oleic acid and sulfonic acids, can also be part of the reaction mixture. Molecular weights of the alkylphenols range from 800 to 2,500. Representative examples are shown in U.S. Pat. Nos. 3,697,574; 3,703,536; 3,704,308; 3,751,365; 3,756,953; 3,798,165; and 3,803,039.

Typical high molecular weight aliphatic acid modified Mannich condensation products useful in this disclosure can be prepared from high molecular weight alkyl-substituted hydroxyaromatics or HNR2 group-containing reactants.

Hydrocarbyl substituted amine ashless dispersant additives are well known to one skilled in the art; see, for example, U.S. Pat. Nos. 3,275,554; 3,438,757; 3,565,804; 3,755,433, 3,822,209, and 5,084,197.

Preferred dispersants include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a Mn of from about 500 to about 5000, or from about 1000 to about 3000, or about 1000 to about 2000, or a mixture of such hydrocarbylene groups, often with high terminal vinylic groups. Other preferred dispersants include succinic acid-esters and amides, alkylphenol-polyamine-coupled Mannich adducts, their capped derivatives, and other related components.

Polymethacrylate or polyacrylate derivatives are another class of dispersants. These dispersants are typically prepared by reacting a nitrogen containing monomer and a methacrylic or acrylic acid esters containing 5 -25 carbon atoms in the ester group. Representative examples are shown in U.S. Pat. Nos. 2, 100, 993, and 6,323,164. Polymethacrylate and polyacrylate dispersants are normally used as multifunctional viscosity modifiers. The lower molecular weight versions can be used as lubricant dispersants or fuel detergents.

Illustrative preferred dispersants useful in this disclosure include those derived from polyalkenyl-substituted mono- or dicarboxylic acid, anhydride or ester, which dispersant has a polyalkenyl moiety with a number average molecular weight of at least 900 and from greater than 1.3 to 1.7, preferably from greater than 1.3 to 1.6, most preferably from greater than 1.3 to 1.5, functional groups (mono- or dicarboxylic acid producing moieties) per polyalkenyl moiety (a medium functionality dispersant). Functionality (F) can be determined according to the following formula:

F=(SAP×Mₙ)/((_{112,200}×x A.I.)-(SAP x 98))

wherein SAP is the saponification number (i.e., the number of milligrams of KOH consumed in the complete neutralization of the acid groups in one gram of the succinic-containing reaction product, as determined according to ASTM D94); Mn is the number average molecular weight of the starting olefin polymer; and A.I. is the percent active ingredient of the succinic-containing reaction product (the remainder being unreacted olefin polymer, succinic anhydride and diluent).

The polyalkenyl moiety of the dispersant may have a number average molecular weight of at least 900, suitably at least 1500, preferably between 1800 and 3000, such as between 2000 and 2800, more preferably from about 2100 to 2500, and most preferably from about 2200 to about 2400. The molecular weight of a dispersant is generally expressed in terms of the molecular weight of the polyalkenyl moiety. This is because the precise molecular weight range of the dispersant depends on numerous parameters including the type of polymer used to derive the dispersant, the number of functional groups, and the type of nucleophilic group employed.

Polymer molecular weight, specifically Mn, can be determined by various known techniques. One convenient method is gel permeation chromatography (GPC), which additionally provides molecular weight distribution information (see W. W. Yau, J. J. Kirkland and D. D. Bly, “Modern Size Exclusion Liquid Chromatography”, John Wiley and Sons, New York, 1979). Another useful method for determining molecular weight, particularly for lower molecular weight polymers, is vapor pressure osmometry (e.g., ASTM D3592).

The polyalkenyl moiety in a dispersant preferably has a narrow molecular weight distribution (MWD), also referred to as polydispersity, as determined by the ratio of weight average molecular weight (Mw) to number average molecular weight (Mn). Polymers having a Mw/Mn of less than 2.2, preferably less than 2.0, are most desirable. Suitable polymers have a polydispersity of from about 1.5 to 2.1, preferably from about 1.6 to about 1.8.

Suitable polyalkenes employed in the formation of the dispersants include homopolymers, interpolymers or lower molecular weight hydrocarbons. One family of such polymers comprise polymers of ethylene and/or at least one C₃ to C₂ alpha-olefin having the formula H₂C=CHR¹ wherein le is a straight or branched chain alkyl radical comprising 1 to 26 carbon atoms and wherein the polymer contains carbon-to-carbon unsaturation, and a high degree of terminal ethenylidene unsaturation. Preferably, such polymers comprise interpolymers of ethylene and at least one alpha-olefin of the above formula, wherein le is alkyl of from 1 to 18 carbon atoms, and more preferably is alkyl of from 1 to 8 carbon atoms, and more preferably still of from 1 to 2 carbon atoms.

Another useful class of polymers is polymers prepared by cationic polymerization of monomers such as isobutene and styrene. Common polymers from this class include polyisobutenes obtained by polymerization of a C₄ refinery stream having a butene content of 35 to 75% by wt., and an isobutene content of 30 to 60% by wt. A preferred source of monomer for making poly-n-butenes is petroleum feed streams such as Raffinate II. These feed stocks are disclosed in the art such as in U.S. Pat. No. 4,952,739. A preferred embodiment utilizes polyisobutylene prepared from a pure isobutylene stream or a Raffinate I stream to prepare reactive isobutylene polymers with terminal vinylidene olefins. Polyisobutene polymers that may be employed are generally based on a polymer chain of from 1500 to 3000.

The dispersant(s) are preferably non-polymeric (e.g., mono- or bis-succinimides). Such dispersants can be prepared by conventional processes such as disclosed in U.S. Patent Application Publication No. 2008/0020950, the disclosure of which is incorporated herein by reference.

The dispersant(s) can be borated by conventional means, as generally disclosed in U.S. Pat. Nos. 3,087,936, 3,254,025 and 5,430,105.

Such dispersants may be used in an amount of about 0.01 to 20 weight percent or 0.01 to 10 weight percent, preferably about 0.5 to 8 weight percent, or more preferably 0.5 to 4 weight percent. Or such dispersants may be used in an amount of about 2 to 12 weight percent, preferably about 4 to 10 weight percent, or more preferably 6 to 9 weight percent. On an active ingredient basis, such additives may be used in an amount of about 0.06 to 14 weight percent, preferably about 0.3 to 6 weight percent. The hydrocarbon portion of the dispersant atoms can range from C₆₀ to C₁₀₀₀, or from C₇₀ to C₃₀₀, or from C₇₀ to C_{200.}These dispersants may contain both neutral and basic nitrogen, and mixtures of both. Dispersants can be end-capped by borates and/or cyclic carbonates. Nitrogen content in the finished oil can vary from about 200 ppm by weight to about 2000 ppm by weight, preferably from about 200 ppm by weight to about 1200 ppm by weight. Basic nitrogen can vary from about 100 ppm by weight to about 1000 ppm by weight, preferably from about 100 ppm by weight to about 600 ppm by weight.

Dispersants as described herein are beneficially useful with the compositions of this disclosure and substitute for some or all of the surfactants of this disclosure. Further, in one embodiment, preparation of the compositions of this disclosure using one or more dispersants is achieved by combining ingredients of this disclosure, plus optional base stocks and lubricant additives, in a mixture at a temperature above the melting point of such ingredients, particularly that of the one or more M-carboxylates (M =H , metal, two or more metals, mixtures thereof).

As used herein, the dispersant concentrations are given on an “as delivered” basis. Typically, the active dispersant is delivered with a process oil. The “as delivered” dispersant typically contains from about 20 weight percent to about 80 weight percent, or from about 40 weight percent to about 60 weight percent, of active dispersant in the “as delivered” dispersant product.

### Detergents

Illustrative detergents useful in this disclosure include, for example, alkali metal detergents, alkaline earth metal detergents, or mixtures of one or more alkali metal detergents and one or more alkaline earth metal detergents. A typical detergent is an anionic material that contains a long chain hydrophobic portion of the molecule and a smaller anionic or oleophobic hydrophilic portion of the molecule. The anionic portion of the detergent is typically derived from an organic acid such as a sulfur-containing acid, carboxylic acid (e.g., salicylic acid), phosphorus-containing acid, phenol, or mixtures thereof. The counterion is typically an alkaline earth or alkali metal. The detergent can be overbased as described herein.

The detergent is preferably a metal salt of an organic or inorganic acid, a metal salt of a phenol, or mixtures thereof. The metal is preferably selected from an alkali metal, an alkaline earth metal, and mixtures thereof. The organic or inorganic acid is selected from an aliphatic organic or inorganic acid, a cycloaliphatic organic or inorganic acid, an aromatic organic or inorganic acid, and mixtures thereof

The metal is preferably selected from an alkali metal, an alkaline earth metal, and mixtures thereof. More preferably, the metal is selected from calcium (Ca), magnesium (Mg), and mixtures thereof.

The organic acid or inorganic acid is preferably selected from a sulfur-containing acid, a carboxylic acid, a phosphorus-containing acid, and mixtures thereof.

Preferably, the metal salt of an organic or inorganic acid or the metal salt of a phenol comprises calcium phenate, calcium sulfonate, calcium salicylate, magnesium phenate, magnesium sulfonate, magnesium salicylate, an overbased detergent, and mixtures thereof.

Salts that contain a substantially stochiometric amount of the metal are described as neutral salts and have a total base number (TBN, as measured by ASTM D2896) of from 0 to 80. Many compositions are overbased, containing large amounts of a metal base that is achieved by reacting an excess of a metal compound (a metal hydroxide or oxide, for example) with an acidic gas (such as carbon dioxide). Useful detergents can be neutral, mildly overbased, or highly overbased. These detergents can be used in mixtures of neutral, overbased, highly overbased calcium salicylate, sulfonates, phenates and/or magnesium salicylate, sulfonates, phenates. The TBN ranges can vary from low, medium to high TBN products, including as low as 0 to as high as 600. Preferably the TBN delivered by the detergent is between 1 and 20. More preferably between 1 and 12. Mixtures of low, medium, high TBN can be used, along with mixtures of calcium and magnesium metal based detergents, and including sulfonates, phenates, salicylates, and carboxylates. A detergent mixture with a metal ratio of 1, in conjunction of a detergent with a metal ratio of 2, and as high as a detergent with a metal ratio of 5, can be used. Borated detergents can also be used.

Alkaline earth phenates are another useful class of detergent. These detergents can be made by reacting alkaline earth metal hydroxide or oxide (CaO, Ca(OH)₂, BaO, Ba(OH)₂, MgO, Mg(OH)₂, for example) with an alkyl phenol or sulfurized alkylphenol. Useful alkyl groups include straight chain or branched C₁-C₃₀ alkyl groups, preferably, C₄-C₂o or mixtures thereof. Examples of suitable phenols include isobutylphenol, 2-ethylhexylphenol, nonylphenol, dodecyl phenol, and the like. It should be noted that starting alkylphenols may contain more than one alkyl substituent that are each independently straight chain or branched and can be used from 0.5 to 6 weight percent. When a non-sulfurized alkylphenol is used, the sulfurized product may be obtained by methods well known in the art. These methods include heating a mixture of alkylphenol and sulfurizing agent (including elemental sulfur, sulfur halides such as sulfur dichloride, and the like) and then reacting the sulfurized phenol with an alkaline earth metal base.

In accordance with this disclosure, metal salts of carboxylic acids are preferred detergents. These carboxylic acid detergents may be prepared by reacting a basic metal compound with at least one carboxylic acid and removing free water from the reaction product. These compounds may be overbased to produce the desired TBN level. Detergents made from salicylic acid are one preferred class of detergents derived from carboxylic acids. Useful salicylates include long chain alkyl salicylates. One useful family of compositions is of the formula

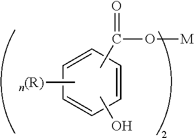

where R is an alkyl group having 1 to about 30 carbon atoms, n is an integer from 1 to 4, and M is an alkaline earth metal. Preferred R groups are alkyl chains of at least C₁₁, preferably C₁₃ or greater. R may be optionally substituted with substituents that do not interfere with the detergent's function. M is preferably, calcium, magnesium, barium, or mixtures thereof. More preferably, M is calcium.

Hydrocarbyl-substituted salicylic acids may be prepared from phenols by the Kolbe reaction (see U.S. Pat. No. 3,595,791). The metal salts of the hydrocarbyl-substituted salicylic acids may be prepared by double decomposition of a metal salt in a polar solvent such as water or alcohol.

Alkaline earth metal phosphates are also used as detergents and are known in the art.

Detergents may be simple detergents or what is known as hybrid or complex detergents. The latter detergents can provide the properties of two detergents without the need to blend separate materials. See U.S. Pat. No. 6,034,039.

Preferred detergents include calcium sulfonates, magnesium sulfonates, calcium salicylates, magnesium salicylates, calcium phenates, magnesium phenates, and other related components (including borated detergents), and mixtures thereof. Preferred mixtures of detergents include magnesium sulfonate and calcium salicylate, magnesium sulfonate and calcium sulfonate, magnesium sulfonate and calcium phenate, calcium phenate and calcium salicylate, calcium phenate and calcium sulfonate, calcium phenate and magnesium salicylate, calcium phenate and magnesium phenate. Overbased detergents are also preferred.

The detergent concentration in the lubricating oils of this disclosure can range from about 0.5 to about 6.0 weight percent, preferably about 0.6 to 5.0 weight percent, and more preferably from about 0.8 weight percent to about 4.0 weight percent, based on the total weight of the lubricating oil.

As used herein, the detergent concentrations are given on an “as delivered” basis. Typically, the active detergent is delivered with a process oil. The “as delivered” detergent typically contains from about 20 weight percent to about 100 weight percent, or from about 40 weight percent to about 60 weight percent, of active detergent in the “as delivered” detergent product.

### Viscosity Modifiers

Viscosity modifiers (also known as viscosity index improvers (VI improvers), and viscosity improvers) can be included in the lubricant compositions of this disclosure.

Viscosity modifiers provide lubricants with high and low temperature operability. These additives impart shear stability at elevated temperatures and acceptable viscosity at low temperatures.

Suitable viscosity modifiers include high molecular weight hydrocarbons, polyesters and viscosity modifier dispersants that function as both a viscosity modifier and a dispersant. Typical molecular weights of these polymers are between about 10,000 to 1,500,000, more typically about 20,000 to 1,200,000, and even more typically between about 50,000 and 1,000,000.

Examples of suitable viscosity modifiers are linear or star-shaped polymers and copolymers of methacrylate, butadiene, olefins, or alkylated styrenes. Polyisobutylene is a commonly used viscosity modifier. Another suitable viscosity modifier is polymethacrylate (copolymers of various chain length alkyl methacrylates, for example), some formulations of which also serve as pour point depressants. Other suitable viscosity modifiers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 molecular weight.

Olefin copolymers are commercially available from Chevron Oronite Company LLC under the trade designation “PARATONE®” (such as “PARATONE® 8921” and “PARATONE® 8941”); from Afton Chemical Corporation under the trade designation “HiTEC®” (such as “HiTEC® 5850B”; and from The Lubrizol Corporation under the trade designation “Lubrizol® 7067C”. Hydrogenated polyisoprene star polymers are commercially available from Infineum International Limited, e.g., under the trade designation “SV200” and “SV600”. Hydrogenated diene-styrene block copolymers are commercially available from Infineum International Limited, e.g., under the trade designation “SV 50”.

The polymethacrylate or polyacrylate polymers can be linear polymers which are available from Evnoik Industries under the trade designation “Viscoplex®” (e.g., Viscoplex 6-954) or star polymers which are available from Lubrizol Corporation under the trade designation Asteric™ (e.g., Lubrizol 87708 and Lubrizol 87725).

Illustrative vinyl aromatic-containing polymers useful in this disclosure may be derived predominantly from vinyl aromatic hydrocarbon monomer. Illustrative vinyl aromatic-containing copolymers useful in this disclosure may be represented by the following general formula:

A—B

wherein A is a polymeric block derived predominantly from vinyl aromatic hydrocarbon monomer, and B is a polymeric block derived predominantly from conjugated diene monomer.

In an embodiment of this disclosure, the viscosity modifiers may be used in an amount of less than about 10 weight percent, preferably less than about 7 weight percent, more preferably less than about 4 weight percent, and in certain instances, may be used at less than 2 weight percent, preferably less than about 1 weight percent, and more preferably less than about 0.5 weight percent, based on the total weight of the formulated oil or lubricating oil. Viscosity modifiers are typically added as concentrates, in large amounts of diluent oil.

As used herein, the viscosity modifier concentrations are given on an “as delivered” basis. Typically, the active polymer is delivered with a diluent oil. The “as delivered” viscosity modifier typically contains from 20 weight percent to 75 weight percent of an active polymer for polymethacrylate or polyacrylate polymers, or from 8 weight percent to 20 weight percent of an active polymer for olefin copolymers, hydrogenated polyisoprene star polymers, or hydrogenated diene-styrene block copolymers, in the “as delivered” polymer concentrate.

### Pour Point Depressants (PPDs)

Conventional pour point depressants (also known as lube oil flow improvers) may be added to the compositions of the present disclosure if desired. These pour point depressant may be added to lubricating compositions of the present disclosure to lower the minimum temperature at which the fluid will flow or can be poured. Examples of suitable pour point depressants include polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. U.S. Pat. Nos. 1,815,022; 2,015,748; 2,191,498; 2,387,501; 2,655, 479; 2,666,746; 2,721,877; 2,721,878; and 3,250,715 describe useful pour point depressants and/or the preparation thereof. Such additives may be used in an amount of about 0.01 to 5 weight percent, preferably about 0.01 to 1.5 weight percent.

### Seal Compatibility Agents

Seal compatibility agents help to swell elastomeric seals by causing a chemical reaction in the fluid or physical change in the elastomer. Suitable seal compatibility agents for lubricating oils include organic phosphates, aromatic esters, aromatic hydrocarbons, esters (butylbenzyl phthalate, for example), and polybutenyl succinic anhydride. Such additives may be used in an amount of about 0.01 to 3 weight percent, preferably about 0.01 to 2 weight percent.

### Antifoam Agents

Anti-foam agents may advantageously be added to lubricant compositions. These agents retard the formation of stable foams. Silicones and organic polymers are typical anti-foam agents. For example, polysiloxanes, such as silicon oil or polydimethyl siloxane, provide antifoam properties. Anti-foam agents are commercially available and may be used in conventional minor amounts along with other additives such as demulsifiers; usually the amount of these additives combined is less than 1 weight percent and often less than 0.1 weight percent.

### Inhibitors and Antirust Additives

Antirust additives (or corrosion inhibitors) are additives that protect lubricated metal surfaces against chemical attack by water or other contaminants. A wide variety of these are commercially available.

One type of antirust additive is a polar compound that wets the metal surface preferentially, protecting it with a film of oil. Another type of antirust additive absorbs water by incorporating it in a water-in-oil emulsion so that only the oil touches the metal surface. Yet another type of antirust additive chemically adheres to the metal to produce a non-reactive surface. Examples of suitable additives include zinc dithiophosphates, metal phenolates, basic metal sulfonates, fatty acids and amines. Such additives may be used in an amount of about 0.01 to 5 weight percent, preferably about 0.01 to 1.5 weight percent.

### Friction Modifiers

A friction modifier is any material or materials that can alter the coefficient of friction of a surface lubricated by any lubricant or fluid containing such material(s). Friction modifiers, also known as friction reducers, or lubricity agents or oiliness agents, and other such agents that change the ability of base oils, formulated lubricant compositions, or functional fluids, to modify the coefficient of friction of a lubricated surface may be effectively used in combination with the base oils or lubricant compositions of the present disclosure if desired. Friction modifiers that lower the coefficient of friction are particularly advantageous in combination with the base oils and lube compositions of this disclosure.

Illustrative friction modifiers may include, for example, organometallic compounds or materials, or mixtures thereof. Illustrative organometallic friction modifiers useful in the lubricating engine oil formulations of this disclosure include, for example, molybdenum amine, molybdenum diamine, an organotungstenate, a molybdenum dithiocarbamate, molybdenum dithiophosphates, molybdenum amine complexes, molybdenum carboxylates, and the like, and mixtures thereof. Similar tungsten based compounds may be preferable.

Other illustrative friction modifiers useful in the lubricating engine oil formulations of this disclosure include, for example, alkoxylated fatty acid esters, alkanolamides, polyol fatty acid esters, borated glycerol fatty acid esters, fatty alcohol ethers, and mixtures thereof

Illustrative alkoxylated fatty acid esters include, for example, polyoxyethylene stearate, fatty acid polyglycol ester, and the like. These can include polyoxypropylene stearate, polyoxybutylene stearate, polyoxyethylene isosterate, polyoxypropylene isostearate, polyoxyethylene palmitate, and the like.

Illustrative alkanolamides include, for example, lauric acid diethylalkanolamide, palmic acid diethylalkanolamide, and the like. These can include oleic acid diethyalkanolamide, stearic acid diethylalkanolamide, oleic acid diethylalkanolamide, polyethoxylated hydrocarbylamides, polypropoxylated hydrocarbylamides, and the like.

Illustrative polyol fatty acid esters include, for example, glycerol mono-oleate, saturated mono-, di-, and tri-glyceride esters, glycerol mono-stearate, and the like. These can include polyol esters, hydroxyl-containing polyol esters, and the like.

Illustrative borated glycerol fatty acid esters include, for example, borated glycerol mono-oleate, borated saturated mono-, di-, and tri-glyceride esters, borated glycerol mono-sterate, and the like. In addition to glycerol polyols, these can include trimethylolpropane, pentaerythritol, sorbitan, and the like. These esters can be polyol monocarboxylate esters, polyol dicarboxylate esters, and on occasion polyoltricarboxylate esters. Preferred can be the glycerol mono-oleates, glycerol dioleates, glycerol trioleates, glycerol monostearates, glycerol distearates, and glycerol tristearates and the corresponding glycerol monopalmitates, glycerol dipalmitates, and glycerol tripalmitates, and the respective isostearates, linoleates, and the like. On occasion the glycerol esters can be preferred as well as mixtures containing any of these. Ethoxylated, propoxylated, butoxylated fatty acid esters of polyols, especially using glycerol as underlying polyol can be preferred.

Illustrative fatty alcohol ethers include, for example, stearyl ether, myristyl ether, and the like. Alcohols, including those that have carbon numbers from C₃ to C₅₀, can be ethoxylated, propoxylated, or butoxylated to form the corresponding fatty alkyl ethers. The underlying alcohol portion can preferably be stearyl, myristyl, C₁₁-C₁₃ hydrocarbon, oleyl, isosteryl, and the like.

The lubricating oils of this disclosure exhibit desired properties, e.g., wear control, in the presence or absence of a friction modifier.

Useful concentrations of friction modifiers may range from 0.01 weight percent to 5 weight percent, or about 0.1 weight percent to about 2.5 weight percent, or about 0.1 weight percent to about 1.5 weight percent, or about 0.1 weight percent to about 1 weight percent. Concentrations of molybdenum-containing materials are often described in terms of Mo metal concentration. Advantageous concentrations of Mo may range from 25 ppm to 700 ppm or more, and often with a preferred range of 50-200 ppm. Friction modifiers of all types may be used alone or in mixtures with the materials of this disclosure. Often mixtures of two or more friction modifiers, or mixtures of friction modifier(s) with alternate surface active material(s), are also desirable.

### Antiwear Additives

A metal alkylthiophosphate and more particularly a metal dialkyl dithio phosphate in which the metal constituent is zinc, or zinc dialkyl dithio phosphate (ZDDP) can be a useful component of the lubricating oils of this disclosure. ZDDP can be derived from primary alcohols, secondary alcohols or mixtures thereof. ZDDP compounds generally are of the formula

Zn[SP(S)(OR¹)(OR²)]₂

where R¹ and R² are C₁-C₁₈ alkyl groups, preferably C₂-C₁₂ alkyl groups. These alkyl groups may be straight chain or branched. Alcohols used in the ZDDP can be propanol, 2-propanol, butanol, secondary butanol, pentanols, hexanols such as 4-methyl-2-pentanol, n-hexanol, n-octanol, 2-ethyl hexanol, alkylated phenols, and the like. Mixtures of secondary alcohols or of primary and secondary alcohol can be preferred. Alkyl aryl groups may also be used.

Preferable zinc dithiophosphates which are commercially available include secondary zinc dithiophosphates such as those available from for example, The Lubrizol Corporation under the trade designations “LZ 677A”, “LZ 1095” and “LZ 1371”, from for example Chevron Oronite under the trade designation “OLOA 262” and from for example Afton Chemical under the trade designation “HITEC 7169”.

The ZDDP is typically used in amounts of from about 0.3 weight percent to about 1.5 weight percent, preferably from about 0.4 weight percent to about 1.2 weight percent, more preferably from about 0.5 weight percent to about 1.0 weight percent, and even more preferably from about 0.6 weight percent to about 0.8 weight percent, based on the total weight of the lubricating oil, although more or less can often be used advantageously. Preferably, the ZDDP is a secondary ZDDP and present in an amount of from about 0.6 to 1.0 weight percent of the total weight of the lubricating oil.

The types and quantities of performance additives used in combination with the instant disclosure in lubricant compositions are not limited by the examples shown herein as illustrations.

When lubricating oil compositions contain one or more of the additives discussed above, the additive(s) are blended into the composition in an amount sufficient for it to perform its intended function. Typical amounts of such additives useful in the present disclosure are shown in Table 1 below.

It is noted that many of the additives are shipped from the additive manufacturer as a concentrate, containing one or more additives together, with a certain amount of base oil diluents. Accordingly, the weight amounts in the table below, as well as other amounts mentioned herein, are directed to the amount of active ingredient (that is the non-diluent portion of the ingredient). The weight percent (wt %) indicated below is based on the total weight of the lubricating oil composition.

TABLE 1
Typical Amounts of Other Lubricating Oil Components

| | Approximate | Approximate |
|---|---|---|
| Compound | wt % (Useful) | wt % (Preferred) |
| Antiwear | 0.1-2 | 0.5-1 |
| Dispersant | 0.1-20 | 0.1-8 |
| Detergent | 0.1-20 | 0.1-8 |
| Antioxidant | 0.1-10 | 0.1-5 |
| Friction Modifier | 0.01-5 | 0.01-1.5 |
| Pour Point Depressant (PPD) | 0.0-5 | 0.01-1.5 |
| Anti-foam Agent | 0.001-3 | 0.001-0.15 |
| Viscosity Index Improver | 0.0-8 | 0.1-6 |
| (pure polymer basis) | | |
| Inhibitor and Antirust | 0.01-5 | 0.01-1.5 |

The foregoing additives are all commercially available materials. These additives may be added independently but are usually precombined in packages which can be obtained from suppliers of lubricant oil additives. Additive packages with a variety of ingredients, proportions and characteristics are available and selection of the appropriate package will take the requisite use of the ultimate composition into account.

ASTM D4172 Four Ball testing can be used to evaluate wear performance of finished lubricants. Under varying load conditions, oils can produce wear scars of <1 mm, or <0.9 mm, or <0.8 mm, or <0.7 mm, or <0.6 mm, or <0.5 mm, or <0.4 mm.

B10 sludge testing can result in sludge ratings of <3, or <2, or <1, or 0.

The following non-limiting examples are provided to illustrate the disclosure.

### EXAMPLES

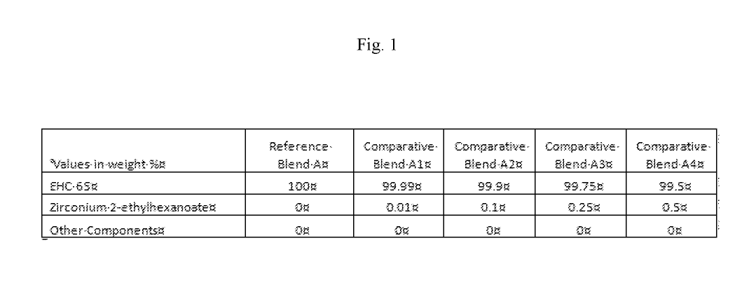

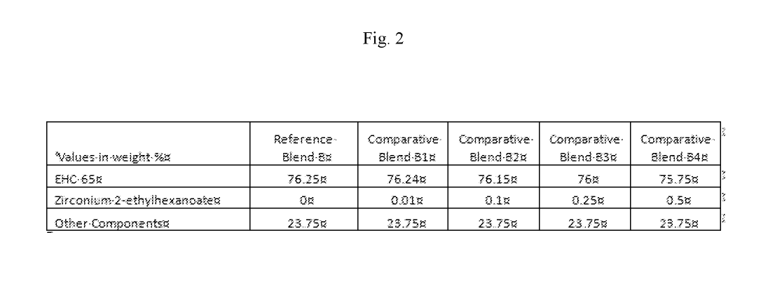

Several lubricating oil candidates were formulated as shown in FIGS. 1 and 2. All of the ingredients used in the candidate formulated oils were commercially available.

Data presented in FIGS. 1 and 2 shows blends made using EHC 65 with varying concentrations of zirconium 2-ethylhexanoate, as well as blends where varying concentrations of zirconium 2-ethylhexanoate are added to a fully formulated diesel engine oil as a top treat.

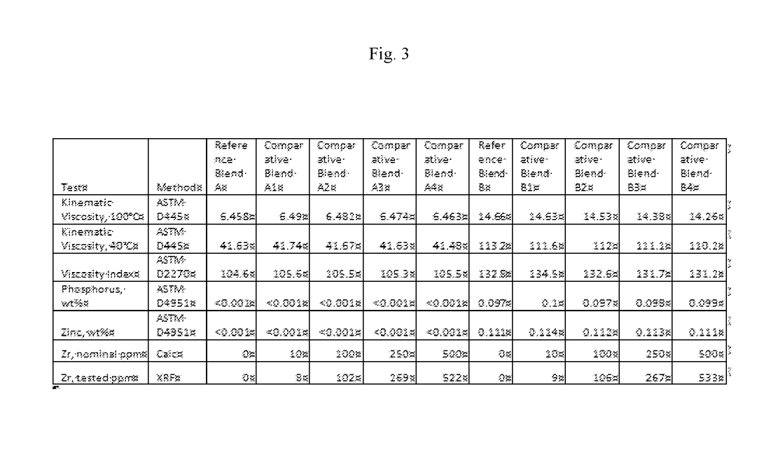

Test results are set forth in FIGS. 3, 4 and 5. Testing includes viscometric properties as shown in FIG. 3, Four Ball Wear Test for wear in accordance with ASTM D4172, and B-10 Catalytic Oxidation Test for sludge.

FIG. 1 shows formulation details for relevant blends in EHC 65. FIG. 1 shows reference oil as neat EHC 65 and comparative blends as varying concentrations of zirconium 2-ethylhexanoate blended with neat EHC 65 base stock.

FIG. 2 shows formulation details for relevant blends in formulated 15W-40 heavy duty engine oil. FIG. 2 shows reference oil as fully formulated commercial 15W-40 heavy duty engine oil and comparative blends as varying concentrations of zirconium 2-ethylhexanoate blended into fully formulated oil in balance with EHC 65 base stock. No other additives changed during blending.

FIG. 3 shows physical properties for relevant blends. FIG. 3 shows viscosity and relevant additive metals in tested formulations.

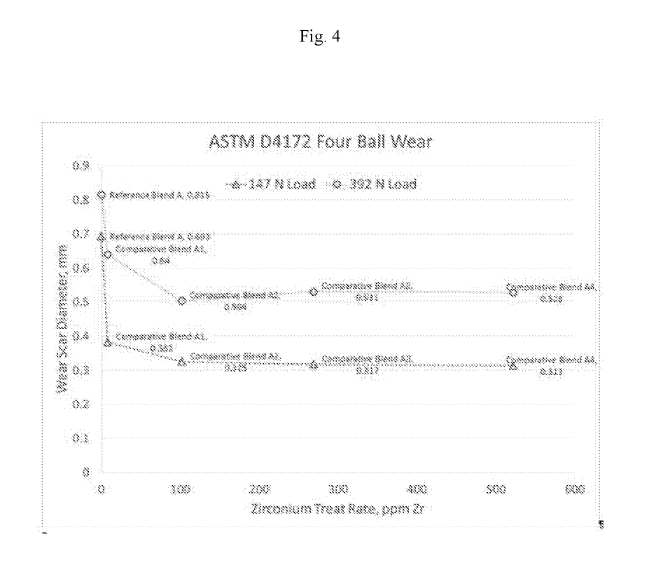

FIG. 4 graphically shows ASTM D4172 Four Ball Wear Test results at medium and high load for reference and comparative blends in neat EHC 65 base stock. The addition of zirconium 2-ethylhexanoate shows a clear and surprising benefit in wear performance as measured in this test, even at very low concentrations. The zirconium treat rate was measured by ppm zirconium delivered by zirconium 2-ethylhexanoate.

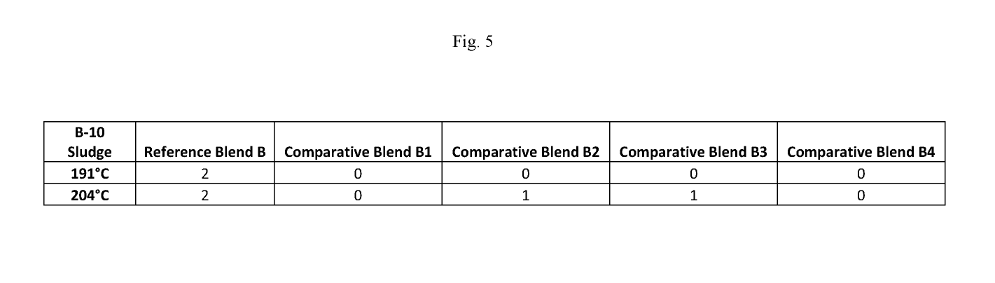

FIG. 5 graphically shows B-10 Sludge Performance by AM/S 334 in Blend Series B. Blend Series B is a fully formulated heavy duty diesel engine oil. The test method AM/S 334 is a high temperature bulk oxidation test, where a sample of oil is catalytically oxidized in a container. After 24 hours, the oil is rated for sludge as Nil, Trace, Light, or Heavy. These ratings were assigned a value of 0 (Nil), 1 (Trace), 2 (Light), or 3 (Heavy) and the results of Blend Series B are shown in FIG. 5. The addition of even small amounts of zirconium surprisingly showed an improvement in sludge performance in this test.

The B-10 Catalytic Oxidation Test for sludge was carried out by subjecting the formulations to a stream of air which was bubbled through at a rate of five liters per hour respectively at 325° F. for 40 hours and/or 375° F. for 24 hours. Present in the formulations were samples of metals commonly used in engine construction, namely, iron, copper, aluminum, and lead. See U.S. Pat. Nos. 3,682,980, 3,445,391, and 5,486,301, the disclosures of which are incorporated herein by reference.

In accordance with this disclosure, unexpected advantages are shown with respect to improving both wear and sludge performance at very low concentrations of a zirconium-containing compound (e.g., zirconium 2-ethylhexanoate). The prior art indicates that wear benefits of zirconium are only obtainable at much higher treat rates, up to 20%. This disclosure describes the ability to improve wear performance with as low as 10 ppm zirconium in the lubricating oil. This disclosure also provides the ability to use the zirconium-containing compound as an oil tracer for fluid identification and anti-counterfitting. Because zirconium is not a common additive metal found in lubricants, and is not a metal typically used in machine components and metallurgy, it is able to be used in this disclosure as a unique product identifier.

### PCT and EP Clauses:

1. A method for improving wear control and sludge control, while maintaining or improving fuel efficiency, of a lubricating oil in an engine or other mechanical component lubricated with the lubricating oil by using as the lubricating oil a formulated oil, said formulated oil having a composition comprising a lubricating oil base stock as a major component; and at least one lubricating oil additive, as a minor component; wherein the at least one lubricating oil additive comprises a zirconium-containing compound; wherein the zirconium-containing compound is present in an amount from 0.1 to 1200 parts per million (ppm); and wherein the zirconium-containing compound is soluble in the lubricating oil base stock.

2. The method of clause 1 wherein, in a Four Ball Wear Test in accordance with ASTM D4172, the wear scar diameter in millimeters (mm) for the lubricating oil having a zirconium treat rate from 0.1 to 1200 parts per million (ppm) is decreased as compared to the wear scar diameter (mm) of a lubricating oil having a zirconium treat rate of 0 ppm.

3. The method of clause 1 wherein, in an engine oil B-10 Catalytic Oxidation Test, the sludge rating for the lubricating oil having a zirconium treat rate from 0.1 to 1200 parts per million (ppm) is decreased as compared to the sludge rating of a lubricating oil having a zirconium treat rate of 0 ppm, wherein the sludge rating is based on a scale of 0 (nil), 1 (trace), 2 (light), and 3 (heavy).

4. The method of clauses 1-3 wherein the zirconium-containing compound is selected from the group consisting of zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, and zirconium salts.

5. The method of clauses 1-4 wherein the zirconium salts are selected from the group consisting of zirconium oleate, zirconium stearate, zirconium palmitate, and zirconium laurate.

6. The method of clauses 1-5 wherein the zirconium-containing compound is present in an amount from 1 to 1000 parts per million (ppm).

7. A lubricating oil having a lubricating oil base stock as a major component, and at least one lubricating oil additive, as a minor component; wherein the at least one lubricating oil additive comprises a zirconium-containing compound; wherein the zirconium-containing compound is present in an amount from 0.1 to 1200 parts per million (ppm); and wherein the zirconium-containing compound is soluble in the lubricating oil base stock.

8. The lubricating oil of clause 7 wherein, in a Four Ball Wear Test in accordance with ASTM D4172, the wear scar diameter in millimeters (mm) for the lubricating oil having a zirconium treat rate from 0.1 to 1200 parts per million (ppm) is decreased as compared to the wear scar diameter (mm) of a lubricating oil having a zirconium treat rate of 0 ppm.

9. The lubricating oil of clause 7 wherein, in an engine oil B-10 Catalytic Oxidation Test, the sludge rating for the lubricating oil having a zirconium treat rate from 0.1 to 1200 parts per million (ppm) is decreased as compared to the sludge rating of a lubricating oil having a zirconium treat rate of 0 ppm, wherein the sludge rating is based on a scale of 0 (nil), 1 (trace), 2 (light), and 3 (heavy).

10. The lubricating oil of clauses 7-9 wherein the zirconium-containing compound is selected from the group consisting of zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, and zirconium salts.

11. The lubricating oil of clauses 7-10 wherein the zirconium salts ate selected from the group consisting of zirconium oleate, zirconium stearate, zirconium palmitate, and zirconium laurate.

12. The lubricating oil of clauses 7-11 wherein the zirconium-containing compound is present in an amount from 1 to 1000 parts per million (ppm).

13. A method for authentication of a lubricating oil, said method comprising: (i) marking the lubricating oil by introducing at least one metallic tracer into the lubricating oil; wherein the lubricating oil comprises a lubricating oil base stock, and the at least one metallic tracer comprises a zirconium-containing compound; wherein the zirconium-containing compound is present in an amount from 0.1 to 1200 parts per million (ppm); and wherein the zirconium-containing compound is soluble in the lubricating oil base stock; (ii) optionally lubricating an engine or other mechanical component with the lubricating oil; and (iii) authenticating the lubricating oil by determining at least one of the identity and amount of the at least one metallic tracer in the lubricating oil.

14. The method of clause 13 wherein the lubricating oil is a used lubricating oil or a non-used lubricating oil.

15. The method of clauses 13 and 14 wherein the authenticating is for product quality control, anti-counterfeit protection, or genuine product verification.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

1. A method for improving wear control and sludge control, while maintaining or improving fuel efficiency, of a lubricating oil in an engine or other mechanical component lubricated with the lubricating oil by using as the lubricating oil a formulated oil, said formulated oil having a composition comprising a lubricating oil base stock as a major component; and at least one lubricating oil additive, as a minor component; wherein the at least one lubricating oil additive comprises a zirconium-containing compound; wherein the zirconium-containing compound is present in an amount from about 0.1 to about 1200 parts per million (ppm); and wherein the zirconium-containing compound is soluble in the lubricating oil base stock.

2. The method of claim 1 wherein, in a Four Ball Wear Test in accordance with ASTM D4172, the wear scar diameter in millimeters (mm) for the lubricating oil having a zirconium treat rate from about 0.1 to about 1200 parts per million (ppm) is decreased as compared to the wear scar diameter (mm) of a lubricating oil having a zirconium treat rate of 0 ppm.

3. The method of claim 1 wherein, in the B-10 Catalytic Oxidation Test, the sludge rating for the lubricating oil having a zirconium treat rate from about 0.1 to about 1200 parts per million (ppm) is decreased as compared to the sludge rating of a lubricating oil having a zirconium treat rate of 0 ppm, wherein the sludge rating is based on a scale of 0 (nil), 1 (trace), 2 (light), and 3 (heavy).

4. The method of claim 1 wherein the zirconium-containing compound is selected from the group consisting of zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, and zirconium salts.

5. The method of claim 4 wherein the zirconium salts are selected from the group consisting of zirconium oleate, zirconium stearate, zirconium palmitate, and zirconium laurate.

6. The method of claim 1 wherein the zirconium-containing compound is present in an amount from about 1 to about 1000 parts per million (ppm).

7. The method of claim 1 wherein the zirconium-containing compound is present in an amount from about 10 to about 800 parts per million (ppm).

8. The method of claim 1 wherein the lubricating oil base stock comprises a Group I, Group II, Group III, Group IV or Group V base oil.

9. The method of claim 1 wherein the lubricating oil base stock is present in an amount from about 70 to about 99 weight percent, based on the total weight of the lubricating oil.

10. The method of claim 1 wherein the formulated oil further comprises one or more of a viscosity modifier, dispersant, detergent, antioxidant, pour point depressant, corrosion inhibitor, metal deactivator, seal compatibility additive, anti-foam agent, inhibitor, and anti-rust additive.

11. The method of claim 1 wherein the formulated oil is free of zinc dialkyl dithio phosphate (ZDDP).

12. The method of claim 1 wherein the lubricating oil is a passenger vehicle engine oil (PVEO), a commercial vehicle engine oil (CVEO) , a hydraulic oil, a gear oil or a transmission oil.

13. A lubricating oil having a lubricating oil base stock as a major component, and at least one lubricating oil additive, as a minor component; wherein the at least one lubricating oil additive comprises a zirconium-containing compound; wherein the zirconium-containing compound is present in an amount from about 0.1 to about 1200 parts per million (ppm); and wherein the zirconium-containing compound is soluble in the lubricating oil base stock.

14. The lubricating oil of claim 13 wherein, in a Four Ball Wear Test in accordance with ASTM D4172, the wear scar diameter in millimeters (mm) for the lubricating oil having a zirconium treat rate from about 0.1 to about 1200 parts per million (ppm) is decreased as compared to the wear scar diameter (mm) of a lubricating oil having a zirconium treat rate of 0 ppm.

15. The lubricating oil of claim 13 wherein, in the B-10 Catalytic Oxidation Test, the sludge rating for the lubricating oil having a zirconium treat rate from about 0.1 to about 1200 parts per million (ppm) is decreased as compared to the sludge rating of a lubricating oil having a zirconium treat rate of 0 ppm, wherein the sludge rating is based on a scale of 0 (nil), 1 (trace), 2 (light), and 3 (heavy).

16. The lubricating oil of claim 13 wherein the zirconium-containing compound is selected from the group consisting of zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, and zirconium salts.

17. The lubricating oil of claim 16 wherein the zirconium salts ate selected from the group consisting of zirconium oleate, zirconium stearate, zirconium palmitate, and zirconium laurate.

18. The lubricating oil of claim 13 wherein the zirconium-containing compound is present in an amount from about 1 to about 1000 parts per million (ppm).

19. The lubricating oil of claim 13 wherein the zirconium-containing compound is present in an amount from about 10 to about 800 parts per million (ppm).

20. The lubricating oil of claim 13 wherein the lubricating oil base stock comprises a Group I, Group II, Group III, Group IV or Group V base oil.

21. The lubricating oil of claim 13 wherein the lubricating oil base stock is present in an amount from about 70 to about 99 weight percent, based on the total weight of the lubricating oil.

22. The lubricating oil of claim 13 further comprising one or more of a viscosity modifier, dispersant, detergent, antioxidant, pour point depressant, corrosion inhibitor, metal deactivator, seal compatibility additive, anti-foam agent, inhibitor, and anti-rust additive.

23. The lubricating oil of claim 13 which is free of zinc dialkyl dithio phosphate (ZDDP).

24. The lubricating oil of claim 13 which is a passenger vehicle engine oil (PVEO), a commercial vehicle engine oil (CVEO), a hydraulic oil, a gear oil or a transmission oil.

25. A method for authentication of a lubricating oil, said method comprising: (i) marking the lubricating oil by introducing at least one metallic tracer into the lubricating oil; wherein the lubricating oil comprises a lubricating oil base stock, and the at least one metallic tracer comprises a zirconium-containing compound; wherein the zirconium-containing compound is present in an amount from about 0.1 to about 1200 parts per million (ppm); and wherein the zirconium-containing compound is soluble in the lubricating oil base stock; (ii) optionally lubricating an engine or other mechanical component with the lubricating oil; and (iii) authenticating the lubricating oil by determining at least one of the identity and amount of the at least one metallic tracer in the lubricating oil.

26. The method of claim 25 wherein the lubricating oil is a used lubricating oil or a non-used lubricating oil.

27. The method of claim 25 wherein the authenticating is for product quality control, anti-counterfeit protection, or genuine product verification.

28. The method of claim 25 wherein, in a Four Ball Wear Test in accordance with ASTM D4172, the wear scar diameter in millimeters (mm) for the lubricating oil having a zirconium treat rate from about 0.1 to about 1200 parts per million (ppm) is decreased as compared to the wear scar diameter (mm) of a lubricating oil having a zirconium treat rate of 0 ppm.

29. The method of claim 25 wherein, in the B-10 Catalytic Oxidation Test, the sludge rating for the lubricating oil having a zirconium treat rate from about 0.1 to about 1200 parts per million (ppm) is decreased as compared to the sludge rating of a lubricating oil having a zirconium treat rate of 0 ppm, wherein the sludge rating is based on a scale of 0 (nil), 1 (trace), 2 (light), and 3 (heavy).

30. The method of claim 25 wherein the zirconium-containing compound is selected from the group consisting of zirconium 2-ethylhexanoate, zirconium octoate, zirconium acetylacetonate, zirconium butoxide, zirconium dibutoxide, zirconium tert-butoxide, bis(cyclopentadienyl)zirconium dihydride, zirconium propoxide, zirconium ethoxide, alkylated zirconium salicylate, alkylated zirconium phenate, alkylated zirconium sulfonate, and zirconium salts.

31. The method of claim 30 wherein the zirconium salts ate selected from the group consisting of zirconium oleate, zirconium stearate, zirconium palmitate, and zirconium laurate.

32. The method of claim 25 wherein the zirconium-containing compound is present in an amount from about 1 to about 1000 parts per million (ppm).

33. The method of claim 25 wherein the zirconium-containing compound is present in an amount from about 10 to about 800 parts per million (ppm).

34. The method of claim 25 wherein the lubricating oil base stock comprises a Group I, Group II, Group III, Group IV or Group V base oil.

35. The method of claim 25 wherein the lubricating oil base stock is present in an amount from about 70 to about 95 weight percent, based on the total weight of the lubricating oil.

36. The method of claim 25 wherein the lubricating oil further comprises one or more of a viscosity modifier, dispersant, detergent, antioxidant, pour point depressant, corrosion inhibitor, metal deactivator, seal compatibility additive, anti-foam agent, inhibitor, and anti-rust additive.

37. The method of claim 25 wherein the lubricating oil is free of zinc dialkyl dithio phosphate (ZDDP).

38. The method of claim 25 wherein the lubricating oil is a passenger vehicle engine oil (PVEO), a commercial vehicle engine oil (CVEO), a hydraulic oil, a gear oil or a transmission oil.
